(19) United States
(12) Plant Patent Application Publication
(10) Pub. No.: US 2015/0026850 P1
(43) Pub. Date: Jan. 22, 2015

(54) Vanda orchid plant 'SPCDW1203'

(50) ;

(71) Applicant: Thumrong Suphachadiwong, Chonburi (TH)

(72) Inventor: Thumrong Suphachadiwong, Chonburi (TH)

(21) Appl. No.: 13/987,374

(22) Filed: Jul. 17, 2013

(51) Int. Cl. A01H 5/00

(52) U.S. Cl. PLT/311

(57) ABSTRACT

A new and distinct Vanda cultivar named ‘SPCDW 1203’ is disclosed, characterized by unique petal coloration of greyed-orange with greyed-red spots and stripes. The plant shows a strong tolerance of cold temperatures, for the Genus. The new variety is a Vanda, typically produced as an indoor ornamental plant.

### LATIN NAME OF THE GENUS AND SPECIES

Vanda hybrid

### VARIETY DENOMINATION

‘SPCDW1203’

### BACKGROUND OF THE INVENTION

This application relates to a new cultivar of Vanda, as described in EU application number 2012/1586, filed Jul. 17, 2012, grant pending, to which applicant claims priority. The new variety is the product of a planned breeding program. The new variety originated as a seedling from the crossing of the unpatented, proprietary seed parent Vanda ‘TKJL’ with the unpatented, proprietary pollen parent referred to as Vanda ‘D2JK’. The crossing was made by the inventor in his research greenhouse in Chonburi, Thailand in June of 2001.

The new variety was first selected by the inventor, Thumrong Suphachadiwong, a citizen of Thailand, in May of 2006, in a research greenhouse belonging to the inventor in Chonburi, Thailand. After identifying the new variety as a potentially interesting selection, the inventor continued confidential testing and propagation of ‘SPCDW1203’, assessing stability of the unique characteristics of this variety.

Asexual reproduction of the new cultivar ‘SPCDW 1203’ was first performed at a commercial laboratory belonging to the inventor in Chonburi, Thailand by tissue culture in June of 2006. Access to all plants was restricted, as plants were kept in a greenhouse not open to the public, and tissue culture plantlets were in a laboratory belonging to the inventor, and not accessible to the public. Through subsequent propagation by vegetative cuttings, multiple generations have been reproduced, which have shown that the unique features of this cultivar are stable and reproduced true to type.

### SUMMARY OF THE INVENTION

The cultivar ‘SPCDW1203’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘SPCDW1203.’ These characteristics in combination distinguish ‘SPCDW1203’ as a new and distinct Vanda cultivar:

1. Unusual greyed-orange petal color with greyed-red spots and stripes.

2. Strong green foliage

3. Good tolerance for cold temperatures, for the Genus

### PARENT COMPARISON

Plants of the new cultivar ‘SPCDW 1203’ are similar to the seed parent, Vanda in most horticultural characteristics. The new variety, however, produces larger flowers than the seed parent.

Plants of the new cultivar ‘SPCDW1203’ are similar to the pollen parent, Vanda in most horticultural characteristics: Plants of the new variety, however, are more compact than the pollen parent.

### COMMERCIAL COMPARISON

‘SPCDW1203’ is similar in most horticultural characteristics to the unpatented commercial variety Vanda ‘Sunanda Magic Gold’ in most horticultural characteristics. The new variety, however, produces sepals and petals colored orange with brown spots, whereas ‘Sunanda Magic Gold’ produces petals and sepals that are yellow with brown spots. The brown spots found on petals and sepals of ‘SPCDW 1203’ are larger than those found on ‘Sunanda Magic Gold’. The new variety has a flower with a different lip shape, and different dorsal sepals than ‘Sunanda Magic Gold’. Additionally, the flower size of the new variety is smaller than that of ‘Sunanda Magic Gold’.

### BRIEF DESCRIPTION OF THE PHOTOGRAPH

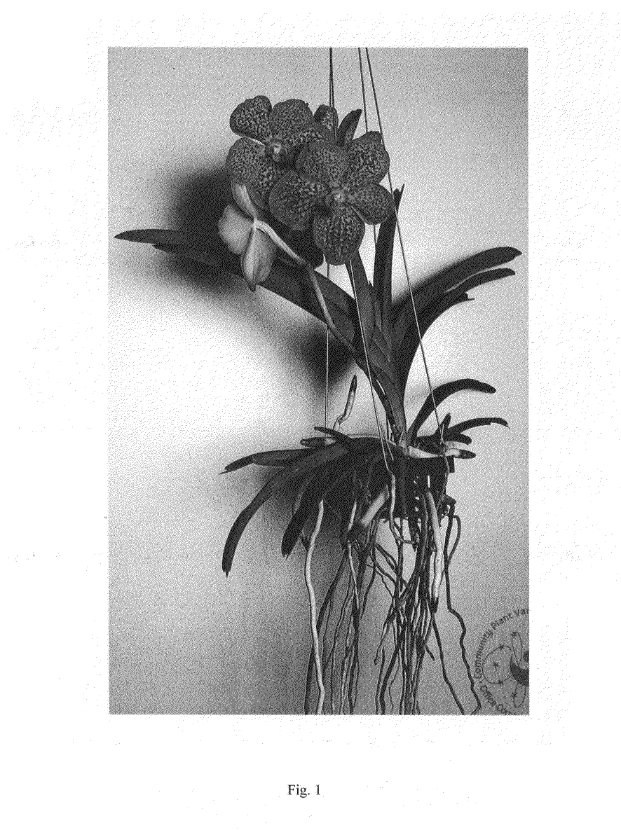

The accompanying photograph in FIG. 1 illustrates in full color a typical plant of ‘SPCDW1203’ grown in a commercial greenhouse in Honselersdijk, the Netherlands. This plant is approximately 3 years old . The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007, except where general terms of ordinary dictionary significance are used. The following observations and measurements describe ‘SPCDW1203’ plants grown in a greenhouse in Honselersdijk, the Netherlands. Temperatures ranged were approximately 23° C. during the day and 19° C. at night. No photoperiodic or chemical treatments were given to the plants. When natural lighting fell below 150 Watt, artificial lighting is given at 10,000 lux for 16 hours a day. Measurements and numerical values represent averages of typical plant types.
Botanical classification: Vanda hybrid ‘SPCDW1203’
Propagation:
   Type of propagation typically used.—Tissue culture.
   Propagation details.—The time needed from for the start of a mericlone to get the small plants ready for deflasking is approximately 24 months under a controlled temperature of about 26 degrees Celsius. After that period plants have matured roots. Plants start to make roots in the last 6 months in flasks. After deflasking they remain in a shaded and controlled environment.
   Root description.—Moderately to heavy rooting with aerial roots only. Roots are very fleshy, non-fibrous. Average length of mature roots: 25 cm Average diameter of mature roots: 2 mm Old roots colored yellow-green: near RHS Grey-Green 196B. Young roots colored greyed-green: near RHS 191 A.
Plant:
   Growth habit.—Monopodial, upright.
   Height from soil line to top of foliage.—Average height from base (plant grown in hanging basket, so no soil line present) to top of foliage: 25 cm.
   Height from soil line to top of flowers.—Average height from base (plant grown in hanging basket, so no soil line present) to top of foliage: 40 cm.
   Plant spread.—Average: 35 cm.
   Normal pot size.—Hanging basket (8×8 cm square),
   Growth rate.—Moderate.
   Number of leaves per plant.—Average: 20.
Foliage: Average length: Approximately 18 cm. Average width: Approximately 2.9 cm. Shape of blade: Ligulate, carinate, slightly twisted lengthways Aspect: Very slightly curved Apex: Unequal emarginated to unequal praemorse Base: Sheathing Sheath length: Average 2.0 cm Sheath width: Average 1.2 cm. Sheath color: Yellow-green; near RHS 144A Margin: Entire Texture of top surface: Smooth, dull Texture of bottom surface: Smooth, dull Color: Young foliage upper side: Near RHS Green 137B Young foliage under side: Near RHS Green 143A Mature foliage upper side: Near RHS Green 137A Mature foliage under side: Near RHS Green 137A Venation: Type: Parallel Venation coloration: Green; near RHS N137B
   Petiole.—No petioles present, leaves sessile.
Flowering characteristics:
   Flower bud.—No flower buds observed at the time of the description.
   Peduncle.—Length: Approximately 25 cm. Width: Approximately 0.5 cm. Aspect: Approximately 20° angle to rosette. Strength: Strong Texture: Smooth, moderately glossy Color: Near RHS Green 143C.
Inflorescence: Flowers per stem: Average: 5 to 7. Inflorescence Arrangement: Axially placed raceme Inflorescence Height,: Approximately 19 cm (measured from base of lowest flower to top of upper flower). Inflorescence Width: Approximately 14 cm.
Individual flowers:
   Flower type.—Zygomorphic (orchidaceous), typical shape for the genus Vanda Shape: Zygomorphic, consisting of three sepals and three petals, one of which is transformed into a labellum (lip). Length (height): Approximately 9 cm. Width: Approximately 11 cm. Depth: Approximately 4.2 cm Fragrance: No fragrance Persistent: No Quantity of Petals: 3 (2 “traditional” petals and a labellum (lip), described separately).
   Petals.—Arrangement: one lateral petal on each side Length: Approximately 5.2 cm. Width: Approximately 4.6 cm. Shape: Broad ovate Apex: Obtuse Base: Cuneate Margin: Entire, wavy Texture: Upper forward surface: Smooth, dull, velvety Texture: Lower back surface: Smooth, dull, velvety Color: When opening, forward surface: Near RHS Greyed-Orange 175B When opening, back surface: Near RHS Greyed-Orange 175D Fully Opened, forward surface: Near RHS Greyed-Orange 164C background color, stripes and spots near Greyed-Orange 175A Fully Opened, back surface: Near RHS Greyed-Orange 164D background color, faint stripes and spots near Greyed-Orange 172A Fading, forward surface: Not fading Fading, back surface: Not fading.
   Labellum/lip.—Shape: Three-lobed, lateral lobes involute, central lobe bi-lobed, with revolute margins Length: Approximately 2 cm Width: Approximately 1.2 cm. Texture: Smooth, dull, with 3 callosities Tip: Bi-lobed Base: Cuneate Color: When opening, forward surface: Near RHS Greyed-Orange 164B, heavily flushed Greyed-Red 178A When opening, back surface: Near RHS Greyed-Orange 164D, heavily flushed Greyed-Red 178A Fully Opened, forward surface: Near RHS Greyed-Orange 164C, heavily flushed Greyed-Red 178A Fully Opened, back surface: Near RHS Greyed-Orange 164D, heavily flushed Greyed-Red 178A Fading, forward surface: Not fading Fading, back surface: Not fading Color Venation Fully Opened, forward surface: No venation visible on labellum Color Venation Fully Opened, back surface: No venation visible on labellum Margin: Entire.
   Spurs.—Quantity: Average: 1 Length: Approximately 0.5 cm. Width: Approximately 0.2 cm. Height: Approximately 0.4 cm. Shape: Short triangular, in line with central lobe of labellum. Apex: Obtuse Base: Broad cuneate Margin: Entire Texture: Smooth, slightly glossy Color: Near RHS Greyed-Red 178C.
   Sepals.—Quantity: Average: 3; One dorsal sepal (pointing upright) and two lateral sepals. Length: Dorsal sepal approximately 5 cm. Lateral sepals approximately 6 cm. Width: Dorsal sepal approximately 5 cm. Lateral sepals approximately 5.5 cm. Shape: Broad ovate Apex: Obtuse Base: Cuneate Margin: Entire, wavy Texture: Smooth, dull Color: Near RHS Greyed-Orange 176B.
   Pedicels.—Length: Approximately 4 cm. Diameter: Approximately 0.5 cm. Angle: Approximately 65° angle to peduncle Strength: Very strong Texture: Smooth, axially angled with 5 “ribs”, axially twisted (due to resupination), glossy Color: Near RHS Greyed-Orange 164C
Reproductive organs: Stamens, style and stigma fused into column? Yes Column Length: Approximately 0.6 cm. Column Diameter: Approximately 0.5 cm. Column Color: Near RHS White 155A Ovary Position: Inferior Diameter: Approximately 0.5 cm. Color: Violet; near 85A Pollinia Color: Near Yellow 15A
Other characteristics:
   Disease resistance.—Good resistance to the normal diseases found in Vanda.
   Drought tolerance and cold tolerance.—Drought tolerance is typical for species. Superior cold tolerance, to approximately 6 degrees Celsius.
   Fruit/seed production.—No fruits/seeds detected to date.

What is claimed is:

1. A new and distinct cultivar of Vanda plant named ‘SPCDW 1203’ as herein illustrated and described.
